(19) United States
(12) Patent Application Publication
(10) Pub. No.: US 2007/0078109 A1
(43) Pub. Date: Apr. 5, 2007

(54) Compositions and methods used to treat acne and candida

(76) Inventors: David Platt, Newton, MA (US); Eliezer Zomer, Newton, MA (US)

Correspondence Address: GREENBERG TRAURIG, LLP, MET LIFE BUILDING, 200 PARK AVENUE, NEW YORK, NY, 10166, US

(21) Appl. No.: 10/597,930

(22) PCT Filed: Feb. 14, 2005
(86) PCT No.: PCT/US05/04430

§ 371 Date: Dec. 4, 2006

Related U.S. Application Data
(60) Provisional application No. 60/544,402, filed on Feb. 13, 2004.

(51) Int. Cl. A61K 31/722

(52) U.S. Cl. 514/55; 514/61

(57) ABSTRACT

Disclosed herein is a water-soluble chelated complex of a co-polymer of N-acetylglucosamine-glucosamine pyrithione sale (AGP). This composition is characterized by a highly viscose texture with excellent surface properties and extremely long acting antimicrobial activity. AGP's antimicrobial activity against acne bacteria has been found superior to the benzyl peroxide the leading acne medication. In addition, AGP has been proven to be effective against many pathogenic yeast. AGP has the potential to be useful as a long acting broad-spectrum antimicrobial composition in a variety of dermatological medicaments and cosmetics.

### RELATED APPLICATIONS

This patent application claims priority to and the benefit of Provisional Patent Application Ser. No. 60/544,402, filed Feb. 13, 2004.

### FIELD OF THE INVENTION

The present invention is directed to the treatment of dermatological disease. In particular, the current invention employs carbohydrate polymers to treat bacterial and fungal based dermatological afflictions.

### BACKGROUND OF THE INVENTION

Acne is a multi-factorial disease that affects the sebaceous (oil-producing) hair follicles (pores) of the skin, primarily on the face and neck, but often on the back and chest as well, where hairs grow most densely. The current understanding of the disease is that increasing levels of androgen sex hormones at puberty lead to increased production of oils and epidermal cells lining the follicles. In its less severe forms, acne is characterized by non-inflammatory papules (comedones, whiteheads or blackheads), which are pores plugged by excessive sebum production and trapped skin cells. In more severe cases, patients develop inflammatory lesions, in which the common and generally non-pathogenic skin bacterium plays a significant role.

According to the American Academy of Dermatology, acne vulgaris affects nearly 100% of adolescents and nearly half of adults over 25 in the U.S. Various sources estimate that 15-40% of Americans seek medical treatment for acne by their mid-teens. Every year, five million prescriptions for oral antibiotics are dispensed for acne treatment in the U.S. Additionally, U.S. prescription and over-the-counter sales of topical acne treatments total approximately $1.1 billion per year.

Although not a serious threat to general health, acne is one of the most socially distressing skin conditions, especially for adolescents, who must deal with a disfiguring disease that erupts just when sexual maturity makes them most sensitive about their appearance. Moreover, severe acne can lead to permanent scarring of the skin that carries the social distress throughout adulthood.

The goal for acne treatment is to clear existing lesions and prevent new ones from occurring. Current topical treatments for acne include benzoyl peroxide, retinoids, salicylic acid and antibiotics such as erythromycin and clindamycin. Broad-spectrum oral antibiotics, retinoids and hormone treatments are also prescribed, although many of these treatments produce undesirable and even dangerous side effects. Topical antibiotics kill off and decrease the population of P. acnes within follicles, as well as reduce the ability of this organism to generate pro-inflammatory molecules.

An important issue in the use of topical antibiotics is the emergence of bacterial resistance and cross-resistance, which can also occur with repeated courses of systemic antibiotics. Currently needed is a topical treatment that is effective against P. acnes and does not induce resistance.

Fungal infections are widely distributed in animal species. The most common agents of fungal infections include various species of the Candida and Aspergillus. The incidence of fungal infections has undergone a significant increase, particularly in humans due to increasing number of patients having impaired immune systems, either as a result of medical therapy for transplant patients or diseases such as AIDS which compromise the immune system. Fungal disease, particularly when systemic, can be life threatening to patients having an impaired immune system or become a chronic diseases effecting patient quality of life.

A number of prior art pharmaceutical agents are commonly used for the treatment of fungal diseases. These materials include compounds such as amphotericin B (AMB), triazoles and flucytosin. AMB is the drug of choice for many systemic fungal infections due to its broad range of activity; however, it is harmful to the kidneys and must be administered intravenously. Many of the triazoles exhibit broad ranging activity and can be administered orally; however, many strains of fungi have become resistant to these materials. Consequently, there is a need for new drugs which are effective in eliminating fungus disease, but are of low toxicity to patients. Ideally, these materials should be simple to prepare, stable, and easy to administer.

### BRIEF SUMMARY OF THE INVENTION

The present invention is directed to the treatment of dermatological disease. In particular, the current invention employs carbohydrate polymers to treat bacterial and fungal based dermatological afflictions.

Compositions described herein can be prepared from available carbohydrate polymers such as chitin or chitosan and chelated together with a naturally occurring broad spectrum antimicrobial agent.

One embodiment of the present invention is directed to a water soluble composition comprising N-acetylglucoseamine (AGA)-glucosamine (GA)-pyrithione (PR), referred to herein as “AGP.” In a particular aspect, AGP has the empirical structural formula: [(NAGA)_{X}, (GA)_{Y}, (PR)_{Z} ]_{N} wherein x is from about 0.01 to about 0.3, y is from about 0.3 to about 0.98 and z is from about 0.01 to about 0.3 molar fractions, wherein x+y+z˜1, and N has a value between about 1 and about 100. In a further aspect, AGP has a molecular weight range of aboutl kDa to about 150 kDa.

Another embodiment of the present invention is directed to methods for treating dermatological pathologies. The etiologic agent for the dermatological pathology can be, for example, bacterial or fungal in origin. In one aspect, an effective amount of a composition of the present invention is administered to a subject in need thereof.

### DETAILED DESCRIPTION OF THE INVENTION

The present invention is directed to compositions and methods used for the treatment of dermatological disease. In particular, the current invention employs carbohydrate polymers to treat bacterial and fungal based dermatological afflictions. Compositions described herein can be prepared from available carbohydrate polymers such as chitin or chitosan and chelated with a naturally occurring broad spectrum antimicrobial agent.

One embodiment of the present invention is directed to a composition of co-polymer of N-acetylglucoseamine (AGA)-glucosamine (GA)-pyrithione (PR), referred to herein as “AGP.” In a particular aspect, AGP has the empirical structural formula: [(NAGA)_{X} (GA)_{Y} (PR)_{Z} ]_{N} wherein x is about 0.01 to about 0.3, y is about 0.3 to about 0.98 and z is about 0.01 to about 0.3 molar fractions having total x+y+z˜1, and N has a value between about 1 and about 100. In a further aspect, AGP has a molecular weight range of about 1 kDa to about 150 kDa.

In accordance with the present invention, AGP can be prepared by thermal and acid hydrolysis of purified chitin. The molecular sizing of the co-polymer can be achieved by either (i) enzymatic treatment such as with endo-chitinases (enzymatic treatment) or (ii) chemically by either peroxide oxidation or reductive alkylation (chemical glycosidic hydrolysis). The parameters of temperature and time are important to achieve the proper ratio of glucosamine and N-acetyl glucosamine, the polymeric building blocks. The co-polymer can also be produced by fractional acetylation of polyglucosamine which is commercially available as USP grade chitosan. The molecular weight of commercially available chitosan typically ranges between about 200 kDa and about 2000 kDa and is usually over 95% deacetylated. Using this reactant, AGP is produced in three steps: (i) acid hydrolysis, (ii) partial acetylation, and (iii) chelation with pyrithione acid or salts. The chelation reaction is performed with timely cycling of pH and temperature, finishing with neutral pH and room temperature. The average molecular weight of the resulting AGP typically is in the range of between about 2 kDa and 120 kDa, more typically in the range of between about 15 kDa and about 80 kDa.

AGP can be prepared through the deacetylation of chitin [_-(1-4)-poly-N-acetyl-D-glucosamine], an abundant natural by-product of the crustacean process industries. It can also be produced from a microbial biomass such as mushroom, fungi and yeast.

One skilled in the art appreciates that chitin is easily obtained from crab or shrimp shells and fungal mycelia. With respect to crustaceans, chitin production is associated with food industries such as shrimp canning. With respect to fungal sources, the production of chitosan-glucan complexes is associated with the fermentation process, similar to those for the production of citric acid from Aspergillus niger, Mucor rouxii, and Streptomyces, which involves alkali treatment yielding chitosan-glucan complexes. The alkali simultaneously removes protein and deacetylates chitin. Depending on the alkali concentration some soluble glycans is removed. The processing of crustacean shells essentially involves the removal of proteins and the dissolution of calcium carbonate that is present in crab shells in high concentrations. The resulting chitin is deacetylated in approximately 40% sodium hydroxide at about 120° C. for about 1-3 h. This treatment produces about 70% deacetylated chitosan.

This compound forms an excellent gel like substance and will form a film upon drying. An example of a carbohydrate surfactant that can be used with the compositions of the present invention is a 0.01% to 1% decyl polyglucose (commercially available from Henkel Corp) has can be added to enhance the formulation's activity. The combination of a broad-spectrum antimicrobial, for example, pyrithione with the carbohydrate co-polymer of the present invention provides the compositions with superior antimicrobial activity again dermal pathogens like, for example, acne and candida. Pyrithione is commercially available as zinc, cupper or sodium pyrithione from ARCH Chemicals Inc., under the trademark OMADINE. It is widely used as an antibacterial and antifungal agent in cosmetics and shampoos.

The pyrithione is chelated by a complex carbohydrate to form AGP. This chelated increases the stability of pyrithione and facilitates binding to a microorganism altering their membrane porosity by interfering with Ca-ATP pumps, thus increasing their susceptibility to the pyrithione. The emulsification property of the AGP provides better penetration into the oily environment of the dermis.

The long term stability of the complex makes AGP a very useful pharmaceutical particularly as an antimicrobial agent in both acute and chronic dermatological diseases such as acne, yeast infections and other general dermatitis.

In preclinical evaluations, the antibacterial activity against P. acnes and various pathogenic candida, AGP demonstrated excellent bactericidal activity including against resistant pathogenic strains.

Activity of AGP Against P. Acnes and Other Pathogens

Studies indicate that P. acnes was completely inhibited by concentration of less than 0.1 mg/mL of AGP. Similarly AGP was active toward Escherichia coli and Staphyloccocus aureus at low concentration.

A study with acne patients using a 4% active AGP formula demonstrated remarkable results in less than 48 hours. The AGP formulation had no irritation or other side effects.

Activitv Against Candida In-vitro and In-vivo

In vitro studies employing AGP against a wide spectrum of pathogenic yeast, including azole-resistant isolates was conducted. Over a dozen pathogenic candida were tested and all demonstrated sensitivity to AGP with a minimum inhibitory concentration (MIC) ranging from 0.05 to 0.7 microgram per milliliter.

Pre-clinical studies with three C. albicans isolates, including one fluconazole-resistant strain, were performed using immunosuppressed animals with cyclophosphamide. Induced infection was treated with a topical application of AGP during a period of 1-7 days. AGP at 0.25% was found to be equivalent in clearing the infection as 2% miconazole, and was free of local adverse effects. See Table 1.

TABLE 1
MlC Results for AGP on a variety of candida pathogenic isolates.
Drug concentration is in g/mL

| Candida | AGP-2.3/0.8 | AGP-4/0.8 | AGP-4/1.6 | AGP-4/2.4 |
|---|---|---|---|---|
| AIbicans 90028 | 0.078 | 0.156 | 0.078 | 0.078 |
| 3153A | 0.156 | | 0.078 | 0.078 |
| 36802 | 0.078 | | | |
| Fluconazole | | | | |
| Resistant | | | | |
| Albicans.- | | | | |
| DT 1413A | 0.312 | | | |
| MF 390 | 0.312 | | | |
| DT 492 | 0.312 | | | |
| DT 740 | 0.312 | | | |
| SZ | 0.312 | | | |
| DT 01-A | 0.312 | | | |
| LF 392.95 | 0.312 | | | |
| LF 412.95 | 0.312 | | | |
| JH 488 | 0.312 | | | |
| DT 1167 A | 0.312 | | | |
| MF-109 | 0.156 | | | |
| LF 360.95 | 0.612 | | | |
| Glabrata | 0.312 | 1.25 | 2.5 | 2.5 |
| 32554 | | | | |
| X-62431 | 0.312 | 0.625 | 5 | 5 |
| 90030 | 0.625 | | 10 | 5 |
| MF 028 | 0.156 | | | |
| MF 037B | 0.312 | | | |
| RI 422 | 0.078 | | | |
| MF 057 | 0.625 | | | |
| Parapsilosis | 0.625 | | 5 | 2.5 |
| 22019 | | | | |
| 90018 | 0.078 | | 0.078 | 0.037 |
| Tropicalis | 0.156 | | | |
| 44508 | | | | |
| JH 782 | 0.312 | | | |
| JH 545A | 0.312 | | | |
| JH 491A | 0.156 | | | |
| JH 780B | 0.312 | | | |
| Krusei 6428 | 0.312 | | | |
| RI 1202 | 0.078 | 0.156 | 0.078 | 0.078 |
| 824A | 0.625 | 2.5 | 5 | 2.5 |
| JH 568 | 2.5 | | | |
| Trichomonas | 2.5-5 | | | |

Any of the identified compounds of the present invention can be administered to a subject, including a human, by itself, or in pharmaceutical compositions where it is mixed with suitable carriers or excipients at doses therapeutically effective to prevent, treat or ameliorate a variety of disorders, including those characterized by that outlined herein. A therapeutically effective dose further refers to that amount of the compound sufficient result in the prevention or amelioration of symptoms associated with such disorders. Techniques for formulation and administration of the compounds of the instant invention may be found in Goodman and Gilman's The Pharmacological Basis of Therapeutics, Pergamon Press, latest edition.

The compounds of the present invention can be targeted to specific sites by direct injection into those sites. Compounds designed for use in the central nervous system should be able to cross the blood-brain barrier or be suitable for administration by localized injection.

Pharmaceutical compositions suitable for use in the present invention include compositions wherein the active ingredients are contained in an effective amount to achieve its intended purpose. More specifically, a therapeutically effective amount means an amount effective to prevent development of or alleviate the existing symptoms and underlying pathology of the subject being treating. Determination of the effective amounts is well within the capability of those skilled in the art.

For any compound used in the methods of the present invention, the therapeutically effective dose can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in humans.

A therapeutically effective dose refers to that amount of the compound that results in the attenuation of symptoms or a prolongation of survival in a subject. Toxicity and therapeutic efficacy of such compounds can be determined by standard pharmaceutical procedures in cell cultures or experimental animals, e.g., for determining the LD₅₀ (the dose lethal to 50% of a given population) and the ED₅₀ (the dose therapeutically effective in 50% of a given population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of a patient's condition. Dosage amount and interval can be adjusted individually to provide plasma levels of the active moiety which are sufficient to maintain the desired effects.

In case of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions of the present invention can be manufactured in a manner that is itself known, e.g., by means of conventional mixing, dissolving, granulating, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention can be formulated in aqueous solutions, preferably in physiologically compatible buffers such as Hank's solution, Ringer's solution, or physiological saline buffer. For transmucosal administration, penetrants appropriate to the barriers to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinyl-pyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage for, e.g., in ampoules or in multidose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspension. Suitable lipohilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations previously described, the compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

A pharmaceutical carrier for the hydrophobic compounds of the invention is a co-solvent system comprising benzyl alcohol, a non-polar surfactant, a water-miscible organic polymer, and an aqueous phase. Naturally, the proportions of a co-solvent system can be varied considerably without destroying its solubility and toxicity characteristics. Furthermore, the identity of the co-solvent components can be varied.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known to those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration can, e.g., include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systematic manner, e.g., via injection of the compound directly into an affected area, often in a depot or sustained release formulation.

Furthermore, one can administer the compound in a targeted drug delivery system, e.g., in a liposome coated with an antibody specific for affected cells. The liposomes will be targeted to and taken up selectively by the cells.

The compositions can, if desired, be presented in a pack or dispenser device which can contain one or more unit dosage forms containing the active ingredient. The pack can, e.g., comprise metal or plastic foil, such as a blister pack. The pack or dispenser device can be accompanied by instruction for administration. Compositions comprising a compound of the invention formulated in a compatible pharmaceutical carrier can also be prepared, placed in an appropriate container, and labeled for treatment of an indicated condition. Suitable conditions indicated on the label can include treatment of a disease such as described herein.

### EXAMPLES

Manufacturing Examples

Chelation of a carbohydrate co-polymer is conveniently carried out by slurrying the carbohydrate co-polymer solution with the desired chelation agents.
a. A slurry was prepared by stirring 1 g of prepared co-polymer (15:85, N-acetylglucosamine to glucosamine, MW 60 kDa—thermally and chemically hydrolyzed chitin) into 25 g deionized water. The pH was adjusted to 5.0 using lactic acid. A 50% solution of pyrithione acid (ARCH chem. Inc.) was added by mixing in 0.25 niL pyrithione acid into the slurry. The mixture was mixed for 1 hour at 37° C. and the pH was increased using IM NaOH to pH 5.5. A 0.1 g of decyl polyglucose (50% solution) was added and slowly mixed for 30 minutes.
b. A slurry was prepared by stirring 1 g of prepared co-polymer (15:85, N-acetylglucosamine to glucosamine, MW 60 kDa—thermally and chemically hydrolyzed chitin) into 25 g deionized water. The pH was adjusted to 5.0 using gluconic acid. A sodium OMADINE® solution (a 40% solution of sodium pyrithione, ARCH Chem. Inc.) at 0.5 mL was added, stirring continue for 1 hour at 37° C. The pH was increased with IM NaOH to pH 5.5. A 0.1 g of decyl polyglucose (50% solution) was added and slow mixing was continued for 30 minutes.
c. A slurry was prepared by stirring 1 g of prepared co-polymer (15:85, N-acetylglucosamine to glucosamine, MW 40 kDa—thermally and hydrogen peroxide hydrolyzed chitin) into 25 g deionized water. The pH was adjusted to 5.0 using gluconic acid. A zinc OMADINE® slurry (a 40% slurry solution of Zinc pyrithione, ARCH Chem. Inc.) at 0.5 mL was added and stirring continued for 1 hour at 37° C. The pH was slowly increased with 1M NaOH to pH 5.5. A 0.1 g of decyl polyglucose (50% solution) was added and slow mixing was continued for 30 minutes.

The durability of these co-polymer complexes was determined by subjecting these complexes to typical end-use conditions such as would be expected of an over the counter device and dermal preparation. A 4% solution was found stable and active after 4 years of storage at room temperature.

What is claimed is:

1. An antimicrobial composition, comprising a combination of a carbohydrate and an antimicrobial agent.

2. The composition of claim 1, wherein said carbohydrate originates from chitan.

3. The composition of claim 2, wherein said carbohydrate is N-acetylglucoseamine-glucosamine.

4. The composition of claim 1, wherein said antimicrobial agent is a broad spectrum antibiotic.

5. The composition of claim 4, wherein said broad spectrum antibiotic is pyrithione.

6. An antimicrobial composition having the following structural formula:
[(NAGA)_{X} (GA)_{Y} (PR)_{Z ]}_{N}
wherein x is from about 0.01 to about 0.3, y is from about 0.3 to about 0.98 and z is from about 0.01 to about 0.3 molar fractions, wherein x+y+z˜1, and N has a value between about 1 and about 100.

7. The composition of claim 6, wherein said composition has a molecular weight in range of about 1 kDa to about 150 kDa.

8. The composition of claim 1, wherein said composition further comprises between about 0.01% to about 1% of decyl polyglucose.

9. A method for treating a dermatological condition, comprising administering to a subject in need thereof an effective amount of a composition having N-acetyl-glucoseamine-glucosamine-pyrithione.

10. The method of claim 9, wherein said dermatological condition is acne or candida.

11. The method of claim 10, wherein said effective amount for treating said acne is 0.1 mg/mL.

12. The method of claim 10, wherein said effective amount for treating said candida is from about 0.05 to about 0.7 microgram/mL.

13. The method of claim 9, wherein said composition further comprises between about 0.01% to about 1% of decyl polyglucose.

14. The method of claim 9, wherein said composition is topically applied to an afflicted region of said subject.

15. A method for treating a dermatological condition comprising administering to a subject in need an effective amount of:
ti [(NAGA)_{X} (GA)_{Y} (PR)_{Z} ]_{N}
wherein x is from about 0.01 to about 0.3, y is from about 0.3 to about 0.98 and z is from about 0.01 to about 0.3 molar fractions, wherein x+y+z˜1, and N has a value between about 1 and about 100.
